(19) United States
(12) Patent Application Publication
(10) Pub. No.: US 2016/0325020 A1
(43) Pub. Date: Nov. 10, 2016

(54) OXYGEN SCAVENGING TISSUE GRAFT WITH ENHANCED REGENERATIVE CAPACITY AND METHOD OF MANUFACTURE THEREOF

(71) Applicant: Mark Schallenberger, Bozeman, MT (US)

(72) Inventor: Mark Schallenberger, Bozeman, MT (US)

(21) Appl. No.: 15/148,585

(22) Filed: May 6, 2016

Related U.S. Application Data
(60) Provisional application No. 62/158,500, filed on May 7, 2015.

(51) Int. Cl. A61L 27/54; A61L 27/14; A61L 27/02; A61L 27/36

(52) U.S. Cl. CPC A61L 27/54; A61L 27/36; A61L 27/14; A61L 27/02; A61L 2300/412; A61L 2430/00

(57) ABSTRACT

The invention relates to a biocompatible, oxygen scavenging tissue graft for repair and regeneration of tissue injury. The oxygen scavenging tissue graft induces a transient, local hypoxic environment that induces the surrounding tissue to upregulate endogenous pro-angiogenic growth factors to enhance the regenerative capacity of the tissue graft and aid in the healing of the tissue injury once the graft is implanted into a host.

### CROSS REFERENCE TO RELATED APPLICATIONS

This application claims priority and the benefit under 35 U.S.C. §119(e) to U.S. Provisional Application Ser. No. 62/158,500, filed on May 7, 2015, which is incorporated by reference in its entirety.

### FIELD OF THE INVENTION

The invention generally relates to a biocompatible, oxygen scavenging tissue graft for repair and regeneration of tissue injury. Upon implantation, the oxygen scavenging tissue graft induces a transient, local hypoxic environment that induces the surrounding tissue to upregulate endogenous pro-angiogenic growth factors to enhance the regenerative capacity of the tissue graft and aid in the healing of the tissue injury.

### BACKGROUND

Tissue grafts are used in a variety of surgical specialties to treat different tissue injuries. They are typically designed to replace, stabilize, supplement, protect, repair, or reinforce damaged tissue or prosthetics. Once implanted, the host typically infiltrates the graft with progenitor cells which in turn remodel and integrate the graft with native host tissue. In many cases, the use of a tissue graft produces better regenerative outcomes than comparable surgeries without the use of a graft.

It is commonly accepted in the field that an insufficient supply of oxygen is the rate limiting step for successful remodeling and integration of tissue grafts. While recipient tissue is naturally programmed to respond to insufficient oxygen supply, angiogenesis induction pathways are typically delayed for several days to several weeks following implantation. This inherent delay can lead to cell death, prevent cellular differentiation, and postpone wound healing. Accordingly, researchers have long sought a method for providing more substantial and faster delivery of oxygen to tissue grafts following their surgical implantation.

Previous research aimed at increasing local oxygen concentration can broadly be separated into three strategies: delivery of exogenous growth factors from tissue grafts, incorporation of oxygen carrying materials into tissue grafts, and incorporation of in situ generating oxygen materials into tissue grafts. An example of delivery of exogenous growth factors from tissue grafts can be found in U.S. Pat. No. 9,012,399 entitled “Controlled Release of growth Factors and Signaling Molecules for Promoting Angiogenesis” to Cao et al. Cao discloses a method of inducing growth of new blood vessels by injecting a device comprising an alginate hydrogel scaffold and vascular endothelial growth factor (VEGF) into a tissue containing mammalian cells. In U.S. Patent Publication No. 2015/0216912 entitled “Methods for Inducing Angiogenesis” to Koob, methods for inducing angiogenesis in a body comprising injecting an effective amount of a solution comprising placental growth factors and stem cells extracted from placental tissue are disclosed. Using the strategy of incorporation of oxygen carrying materials into tissue grafts, U.S. Patent Publication No. 2003/0190367 entitled “Oxygen Enriched Implant for Orthopedic Wounds and Method of Packaging and Use” to Balding discloses a composition for bone injury repair comprising a bone void filling material and an oxygen supply material. In other embodiments, the oxygen supply material comprises a perfluoronated hydrocarbon. Balding further discloses a method of packaging the composition, which involves pressurization of the storage container with oxygen. U.S. Patent Publication No. 2012/0082704 entitled “Oxygenated Demineralized Bone Matrix for Use in Bone Growth” to

Phillips et al. discloses a composition comprising an oxygen carrier and demineralized bone matrix. In further embodiments, the oxygen carrier is a perfluorocarbon. Phillips also discloses a method of inducing bone growth comprising mixing an oxygen carrier and DBM and implanting into a patient. Incorporation of in situ generating oxygen materials into tissue grafts are disclosed in U.S. Patent Publication No. 2010/0112087 to Harrison et al. entitled “Oxygen-Generating Compositions for Enhancing Cell and Tissue Survival In Vivo.” Harrison et al. disclose a method of treating hypoxic tissue comprising contacting said tissue with a composition comprising a biodegradable polymer and an inorganic peroxide incorporated into said polymer in solid form.

However, each of these strategies possesses substantial limitations. Delivery of exogenous growth factors is expensive, technically challenging, and subject to stringent regulatory hurdles. Incorporation of oxygen carrying or oxygen generating materials appears logical, but may actually harm the long-term success of the tissue grafts. It is well documented that hypoxia is a critical step in natural pro-angiogenic induction pathways. Through the early delivery of oxygen, these hypoxia inducible angiogenic pathways would be suppressed leading to a delay in implant vascularization that is needed for long term tissue graft remodeling and integration.

### SUMMARY OF THE INVENTION

The invention disclosed herein provides a tissue graft with enhanced regenerative capacity via increasing the expression of native pro-angiogenic growth factors in the host tissue surrounding the tissue graft. In certain embodiments of the present invention, a biocompatible oxygen scavenger is combined with a tissue graft. Once implanted, the oxygen scavenger induces a transient, local hypoxic environment surrounding the tissue graft to enhance the expression of native pro-angiogenic growth factors. This up regulation of native pro-angiogenic growth factors leads to faster vascularization and thus more rapid oxygen delivery to the tissue surrounding the tissue graft. In some embodiments of the invention, the tissue graft can be a human bone allograft. In some embodiments, the tissue graft can be a demineralized bone matrix. In some embodiments of the invention, the biocompatible oxygen scavenger can be a perfluorocarbon compound. In some embodiments of the invention, the perfluorocarbon compound can be deoxygenated. Furthermore, aspects of the invention include methods for preparing the combination of a tissue graft and a biocompatible oxygen scavenger.

An aspect of the invention is an implant for repairing tissue injury. The implant includes a tissue graft and a biocompatible oxygen scavenger.

An aspect of the invention is a method of preparing a composition for repairing tissue injury with enhanced regenerative capacity comprising combining a tissue graft with a biocompatible oxygen scavenger.

### DETAILED DESCRIPTION OF THE INVENTION

The invention relates to an implant for repair of tissue injury with enhanced regenerative capabilities. The implants are comprised of a tissue graft and a biocompatible oxygen scavenger. The oxygen scavenger induces a transient, local hypoxic environment in host tissue surrounding the tissue graft following implantation thus inducing the expression of native pro-angiogenic growth factors. This increased expression of native pro-angiogenic growth factors leads to enhanced tissue repair by increasing vascularization surrounding the tissue graft. The invention also provides preferred method embodiments for production of the implants.

“Tissue graft” as used herein, is an implant of synthetic, biological, or a combination of synthetic and biological origin employed surgically to replace, stabilized, supplement, protect, repair, or reinforce damaged tissue or prosthetics.

“Biocompatible” as used herein, is defined as the quality of being non-toxic to host tissue, cells, proteins, or molecules.

“Enhanced regenerative capacity” as used herein, is defined as a characteristic of a tissue graft that therapeutic benefit is provided either faster or more completely than a different tissue graft.

“Hypoxia” as used herein, is defined as a condition in which a region of tissue is deprived of adequate oxygen supply.

“Allograft tissue” is defined as a tissue derived from a non-identical donor of the same species. “Autograft tissue” is defined as tissue derived from and implanted into the same identical patient.

“Xenograft tissue” is defined as tissue derived from a non-identical donor of a different species.

“Oxygen scavenger” as used herein, is a molecule or compound that possesses affinity for molecular oxygen.

“Pro-angiogenic growth factors” as used herein, is defined as signaling molecules or proteins that promote vascularization through the formation of new blood vessels from pre-existing vessels or from the de novo formation of blood vessels.

Examples of tissue grafts of biological origin broadly include, but are not limited to, allograft tissue, autograft tissue and xenograft tissue. Examples of these types of biological tissues broadly include, but are not limited to, cortical bone, cancellous bone, demineralized bone, connective tissue, tendon, ligaments, pericardium, dermis, cornea, dura matter, fascia, heart valve, veins, artery, ligament, capsular graft, cartilage, collagen, nerve, placental tissue, and combinations thereof.

Examples of tissue grafts of synthetic origin broadly include, but are not limited to, implants manufactured of plastic, metal, thermoplastics, elastomers, polymers, minerals, organic minerals, and combinations thereof. Suitable polymers include, but are not limited to, polycaprolactones, polyethylene glycols, polyhydroxyalkanoates, polyesteramides, polyglycolides, polylactides, polyorthoesters, polyoxazolines, polyurethanes, polylactide-co-glycolides and combinations, and copolymers thereof. Suitable plastics, elastomers, metals, minerals, and organic minerals include, but are not limited to, medical grade PVC, polyethylene, PEEK, polycarbonate, polypropylene, polysulfone, polyurethane, silicone, Grade V titanium, 316LV stainless steel, calcium phosphate, calcium sulfate, hydroxyapatite and combinations thereof.

In some embodiments, the biological tissue used in this invention is selected from the group of human allografts. In some embodiments the human allograft can be human bone. In some embodiments the human bone can be demineralized. In some embodiments the demineralized bone can be combined with a carrier material to aid in the handling or performance of the graft.

It is desirable for the biological tissues of the invention to be decellularized to reduce immunological response in the recipient of the implant. Methods for biological tissue decellularization can be accomplished with materials, which include, but are not limited to, detergents, solvents, acids, bases and combinations thereof, and/or with methods, including but not limited to, freeze-thaw cycling, sonication, irradiation and combinations of the foregoing.

Additionally, the biological tissue can be subjected to one or more additional processing steps commonly known by one skilled in the art. Examples of processing steps include, but are not limited to, disinfection, freezing, lyophilization, cleaning, rinsing, stabilization, packaging, sterilization, and combinations thereof.

In some embodiments, the oxygen scavenger possesses more affinity for molecular oxygen than the native host tissue where the tissue graft is implanted. In some embodiments, the oxygen scavenger can pull molecular oxygen from the host tissue surrounding the implant producing a transient hypoxic environment surrounding the graft. In some embodiments, the oxygen scavenger can be deoxygenated prior to use. Methods of deoxygenation can include placement of the graft in a vacuum or reduced pressure environment, placement of the graft in an oxygen-poor or fully deoxygenated atmosphere, and/or combinations of the foregoing. In some embodiments, the oxygen scavenger can be absorbed by the host following implantation. In some embodiments, the oxygen scavenger can be metabolized by the host following implantation. In some embodiments, the oxygen scavenger can be excreted by the host following implantation.

The oxygen scavenger can be of biological or synthetic origin. Suitable oxygen scavengers of biological origin include, but are not limited to, heme-based formulations, hemoglobin-based formulations, myoglobin-based formulations and combinations thereof Suitable oxygen scavengers of synthetic origin include, but are not limited to, perfluorooctyl bromide, perfluorohexyl bromide, perfluorooctane, perfluoropentane, perfluorohexane, perfluorodecalin, perfluorotributylamine (and salts thereof), perfluorotriisopropylamine (and salts thereof), perfluoro-crown ethers (containing 12, 15, or 18 crown ethers) or combinations thereof. Preferably, the oxygen scavenger can be deoxygenated prior to use to optimize its oxygen scavenging propensity.

Tissue hypoxia has previously been identified as one of the major conditions that result in the expression or increased expression of pro-angiogenic growth factors to increase vascularity to the hypoxic region. In some embodiments of the present invention the hypoxic state in the surrounding tissue ranges from complete to partial depletion of oxygen supply. The hypoxic state can be induced for a period of time required to induce the expression of pro-angiogenic growth factors. In some embodiments the hypoxic state can be induced for a period of time ranging between about 1 second to about 5 weeks, about 1 minute to about 5 weeks, about 1 second to about 20 days, or about 1 minute to about 20 days.

Example of pro-angiogenic growth factors include, but are not limited to, vascular endothelial growth factors (VEGF), fibroblast growth factors, angiopoeitin 1, angiopoietin 2, platelet-derived growth factor, transforming growth factor beta, ephrins, vascular endothelial cadherin or combinations thereof. In some embodiments, the expression of the pro-angiogenic growth factors can be increased by at least 10% compared to expression levels in host tissue exposed to a tissue graft without an oxygen scavenger. The expression of other growth factors can also be increased by the presence of the oxygen scavenging tissue graft. These growth factors include, but are not limited to, bone morphogenetic proteins, colony-stimulating factors, epidermal growth factor, fibroblast growth factor, and insulin-like growth factors.

The biocompatible oxygen scavenger and tissue graft can be combined neat or in solution. The resulting composition can be homogeneous or heterogeneous in regards to the oxygen scavenger and the tissue graft. The biocompatible oxygen scavenger can be present in concentrations ranging from about 0.1% to about 99% by weight of the tissue graft. In some embodiments, the concentration of the biocompatible oxygen scavenger can be between about 5% and 80% by weight of the tissue graft, in some embodiments between about 10% to about 20% by weight of the tissue graft. The biocompatible oxygen scavenger can be passively, ionically, covalently bound or a combination of passively, ionically, and covalently bound to the tissue graft.

Alternatively, the biocompatible oxygen scavenger can be coated on the tissue graft. Methods of coating the tissue graft with the oxygen scavenger include first preparing a composition of oxygen scavenger comprising a solution, suspension, slurry, emulsions, paste, gel or combination thereof. Following preparation of the composition of oxygen scavenger, the implants can be applied to the composition by spraying, dipping, rolling, painting, or other suitable method. Alternatively, the oxygen scavenger can be applied as a paste or powder to the exterior surface of the implant.

Following implantation, the biocompatible oxygen scavenger can be absorbed, excreted metabolized, and/or otherwise removed by the host tissue. The oxygen scavenger can be preferentially removed by the host tissue at a time period following the tissue up regulation of pro-angiogenic growth factors. In some embodiments, this time period can be several minutes to several weeks. In some embodiments, the time period can be between about one minute and about 6 weeks, in some embodiments between about 10 minutes and about 5 weeks.

The coating compositions of oxygen scavenger can also contain biodegradable polymers. Following the coating of the tissue graft with the oxygen scavenger, the resultant coating can be dried by evaporation, heating, lyophilization, similar methods or combinations thereof. In some embodiments, following coating of the tissue graft implant, the implant can be dried and then stored frozen. In some embodiments, following coating of the implant, the implant can be left undried and then stored frozen. In some embodiments, the oxygen scavenger can be gradually released by the biodegradable polymer following implantation. The gradual release can be achieved by passive diffusion of the oxygen scavenger through the biodegradable polymer, through the degradation of the biodegradable polymer or by a combination of the two.

In some embodiments, an additional carrier material can be added to the tissue graft and the oxygen scavenger to aid in handling or the performance of the graft. Suitable carrier materials include, but are not limited to, carboxymethylcellulose, hyaluronate, starch, collagen, polyethylene oxide, lecithin lipids, alginate, gelatin, calcium based salts, or combinations thereof.

An aspect of the invention is an implant for repairing tissue injury. The implant includes a tissue graft and a biocompatible oxygen scavenger.

The tissue graft can be of a biological origin. For example, the tissue graft of the biological origin can be selected from the group consisting of a cortical bone, a cancellous bone, a demineralized bone, a partially demineralized bone, a connective tissue, a tendon, a pericardium, dermis, a cornea, a dura matter, a fascia, a heart valve, a ligament, a capsular graft, cartilage, a collagen, a nerve, a placental tissue, and combinations thereof. The tissue graft can be of synthetic origin. Suitable tissue graft of synthetic origin can be selected from the group consisting of a metal, a thermoplastic, an elastomer, a polymer, a mineral, an organic mineral, and combinations thereof.

The biocompatible oxygen scavenger can be present in concentrations ranging from about 0.1% to about 99% by weight of the tissue graft. In some embodiments, the concentration of the biocompatible oxygen scavenger can be between about 5% and 80% by weight of the tissue graft, in some embodiments between about 10% and about 20% by weight of the tissue graft. In some embodiments, the biocompatible oxygen scavenger can be deoxygenated. The concentration of dissolved oxygen in the biocompatible oxygen scavenger of the deoxygenated graft is defined as reduction in oxygen content from the level of oxygen in a sample exposed to the earth's atmosphere (about 78% nitrogen, 21% oxygen, trace other gases). The concentration of dissolved oxygen in the deoxygenated biocompatible oxygen scavenger component of the tissue graft is anticipated to be reduced by about 20% to about 90% from the initial oxygen concentration of the oxygen scavenger. Methods to quantify the amount of dissolved oxygen may be selected from the following group: colorimetric, titrimetric, and polarographic. The biocompatible oxygen scavenger can be of biological origin. Suitable biocompatible oxygen scavengers can be selected from the group consisting of a heme-based formulation, a hemoglobin-based formulation, and a myoglobin-based formulation, and combinations thereof. The biocompatible oxygen scavenger can be of synthetic origin. Suitable biocompatible oxygen scavengers of synthetic origin can include a perfluorocarbon. Suitable perfluorocarbons can include at least one of a perfluorooctyl bromide, a perfluorohexyl bromide, a perfluorooctane, a perfluoropentane, a perfluorohexane, a perfluorodecalin, a perfluorotributylamine, a salt of perfluorotributylamine, a perfluorotriisopropylamine, a salt of perfluorotriisopropylamine, a perfluoro-crown ether containing 12 crown ethers, a perfluoro-crown ether containing 15 crown ethers, and a perfluoro-crown ether containing 18 crown ethers.

Without being bound by theory, the oxygen scavenger can induce transient hypoxia in the tissue surrounding the implant upon implantation. Without being bound by theory, the transient hypoxia in the tissue surrounding the implant can induce expression of pro-angiogenic growth factors in the tissue surrounding the implant.

An aspect of the invention is a method of preparing a composition for repairing tissue injury with enhanced regenerative capacity comprising combining a tissue graft with a biocompatible oxygen scavenger.

The tissue graft can be of a biological origin. For example, the tissue graft of the biological origin can be selected from the group consisting of a cortical bone, a cancellous bone, a demineralized bone, a partially demineralized bone, a connective tissue, a tendon, a pericardium, dermis, a cornea, a dura matter, a fascia, a heart valve, a ligament, a capsular graft, cartilage, a collagen, a nerve, a placental tissue, and combinations thereof. The tissue graft can be of synthetic origin. Suitable tissue graft of synthetic origin can be selected from the group consisting of a metal, a thermoplastic, an elastomer, a polymer, a mineral, an organic mineral, and combinations thereof.

The biocompatible oxygen scavenger can be present in concentrations ranging from about 0.1% to about 99% by weight of the tissue graft. In some embodiments, the concentration of the biocompatible oxygen scavenger can be between about 5% and 80% by weight of the tissue graft, in some embodiments between about 10% and about 20% by weight of the tissue graft. In some embodiments, the biocompatible oxygen scavenger can be deoxygenated. The concentration of dissolved oxygen in the biocompatible oxygen scavenger of the deoxygenated graft is defined as reduction in oxygen content from the level of oxygen in a sample exposed to the earth's atmosphere (about 78% nitrogen, 21% oxygen, trace other gases). The concentration of dissolved oxygen in the deoxygenated biocompatible oxygen scavenger component of the tissue graft is anticipated to be reduced by about 20% to about 90% from the initial oxygen concentration of the oxygen scavenger. Methods to quantify the amount of dissolved oxygen may be selected from the following group: colorimetric, titrimetric, and polarographic. The biocompatible oxygen scavenger can be of biological origin. Suitable biocompatible oxygen scavengers can be selected from the group consisting of a heme-based formulation, a hemoglobin-based formulation, and a myoglobin-based formulation, and combinations thereof. The biocompatible oxygen scavenger can be of synthetic origin. Suitable biocompatible oxygen scavengers of synthetic origin can include a perfluorocarbon. Suitable perfluorocarbons can include at least one of a perfluorooctyl bromide, a perfluorohexyl bromide, a perfluorooctane, a perfluoropentane, a perfluorohexane, a perfluorodecalin, a perfluorotributylamine, a salt of perfluorotributylamine, a perfluorotriisopropylamine, a salt of perfluorotriisopropylamine, a perfluoro-crown ether containing 12 crown ethers, a perfluoro-crown ether containing 15 crown ethers, and a perfluoro-crown ether containing 18 crown ethers. In some embodiments, the biocompatible oxygen scavenger of the composition can be deoxygenated.

### EXAMPLES

Preparation of a tissue graft containing a biocompatible oxygen scavenger. Combining a tissue graft with a biocompatible oxygen scavenger can be accomplished by numerous routes. Any of the following routes can include other bioactive agents as desired including, but not limited to, antibiotics, growth factors, cells, and biocompatible minerals.

### Example 1

This example illustrates the preparation of a tissue graft of the present invention.

A tissue graft is placed into an emulsion, suspension, slurry, or gel containing a biocompatible oxygen scavenger or mixture of biocompatible oxygen scavengers for a period of time. The emulsion, suspension, slurry, or gel can contain other bioactive agents including, but not limited to, antibiotics, growth factors, cells, and biocompatible minerals. The tissue graft, coated or embedded with the biocompatible oxygen scavenger(s) is removed. This preparation of the implant material is performed at some time prior to the surgical intervention or immediately prior to implantation.

### Example 2

This example illustrates the preparation of a tissue graft of the present invention.

A neat solution of biocompatible oxygen scavenger or mixture of biocompatible oxygen scavengers is injected onto or into the tissue graft. This preparation of the implant material is performed at some time prior to the surgical intervention or immediately prior to implantation.

### Example 3

This example illustrates the preparation of a tissue graft of the present invention.

A tissue graft is placed in a biocompatible oxygen scavenger suspension, gel, slurry, or emulsion, and then the combination of tissue graft and biocompatible oxygen scavenger(s) is frozen. The implant material is then be thawed prior to surgical implantation.

### Example 4

This example illustrates the preparation of a tissue graft of the present invention.

The tissue graft is placed in a biocompatible oxygen scavenger suspension, gel, slurry, or emulsion, and then the combination of tissue graft and biocompatible oxygen scavenger(s) is frozen. This frozen combination is then lyophilized to increase the stability of the composition.

The implant material is then be rehydrated prior to implantation. Suitable rehydration solutions include, but are not limited to, aqueous buffers and biocompatible water miscible solvents.

### Example 5

This example illustrates the preparation of a tissue graft of the present invention.

A tissue graft is placed in a biocompatible oxygen scavenger suspension, gel, slurry, or emulsion, and then the liquid carriers of the biocompatible oxygen scavenger are thoroughly perfused through the tissue graft by vacuum. The tissue graft coated or embedded implant is then (a) stored for later use, (b) used immediately for surgical implantation, (c) frozen and later thawed for use, or (d) frozen, lyophilized, stored, and rehydrated prior to use.

### Example 6

This example illustrates the preparation of a tissue graft of the present invention.

A tissue graft is coated with the biocompatible oxygen scavenger as a neat solution, suspension, gel, slurry or emulsion by a plasma treatment process. The plasma process can consist of direct surface functionalization of the tissue graft substitute with the biocompatible oxygen scavenger, activation of the surface of the tissue graft by initial plasma treatment with a small organic molecule followed by plasma treatment with the contrast agent, activation of the surface of the tissue graft by initial plasma treatment with a small organic molecule followed by placement of the now plasma treated tissue graft into a neat solution, suspension, gel, slurry or emulsion of the biocompatible oxygen scavenger, or other plasma treatment techniques known in the existing art. The plasma coated tissue graft is then (a) stored for later use, (b) used immediately for surgical implantation, (c) frozen and later thawed for use, or (d) frozen, lyophilized, stored, and rehydrated prior to use.

### Methods of Use

### Example 7

This example illustrates a method of use of the tissue graft.

The surgical implant fabricated according to the routes listed in Example 1-6 is implanted into a tissue void or defect. Following and during implantation, the implant serves to temporarily decrease the oxygen content within the tissue void or defect. The transient hypoxia in the tissue serves to induce the expression of native pro-angiogenic growth factors in the tissue surrounding the implant. The increased production of pro-angiogenic growth factors enhances the regenerative capacity of the implant.

### Example 8

This example illustrates a method of use of the tissue graft.

The surgical implant fabricated according to the routes listed in Example 1-6 is deoxygenated prior to use. Methods of deoxygenation can include subjection of the implant to a vacuum and/or an oxygen-depleted atmosphere. Following deoxygenation, the implant can be implanted into a tissue void or defect. Following and during implantation, the implant serves to temporarily decrease the oxygen content within the tissue void or defect. The transient hypoxia in the tissue serves to induce the expression of native pro-angiogenic growth factors in the tissue surrounding the implant. The increased production of pro-angiogenic growth factors enhances the regenerative capacity of the implant.

Ranges have been discussed and used within the forgoing description. One skilled in the art would understand that any sub-range within the stated range would be suitable, as would any number within the broad range, without deviating from the invention.

The foregoing description of the present invention has been presented for purposes of illustration and description. Furthermore, the description is not intended to limit the invention to the form disclosed herein. Consequently, variations and modifications commensurate with the above teachings, and the skill or knowledge of the relevant art, are within the scope of the present invention. The embodiment described hereinabove is further intended to explain the best mode known for practicing the invention and to enable others skilled in the art to utilize the invention in such, or other, embodiments and with various modifications required by the particular applications or uses of the present invention. It is intended that the appended claims be construed to include alternative embodiments to the extent permitted by the prior art.

1. An implant for repairing tissue injury comprising:
a tissue graft; and
a biocompatible oxygen scavenger.

2. The implant of claim 1, wherein the tissue graft is of a biological origin.

3. The implant of claim 2, wherein the tissue graft of the biological origin is selected from the group consisting of a cortical bone, a cancellous bone, a demineralized bone, a partially demineralized bone, a connective tissue, a tendon, a pericardium, dermis, a cornea, a dura matter, a fascia, a heart valve, a ligament, a capsular graft, cartilage, a collagen, a nerve, a placental tissue, and combinations thereof.

4. The implant of claim 1, wherein the tissue graft is of synthetic origin.

5. The implant of claim 4, wherein the tissue graft of synthetic origin is selected from the group consisting of a metal, a thermoplastic, an elastomer, a polymers, a mineral, an organic mineral, and combinations thereof

6. The implant of claim 1, wherein the biocompatible oxygen scavenger has been deoxygenated.

7. The implant of claim 1, wherein the biocompatible oxygen scavenger is of biological origin.

8. The implant of claim 7, wherein the biocompatible oxygen scavenger of biological origin is selected from the group consisting of a heme-based formulation, a hemoglobin-based formulation, and a myoglobin-based formulation.

9. The implant of claim 1, wherein the biocompatible oxygen scavenger is of synthetic origin.

10. The implant of claim 9, wherein the biocompatible oxygen scavenger of synthetic origin is a perfluorocarbon.

11. The implant of claim 10, wherein the perfluorocarbon is at least one of a perfluorooctyl bromide, a perfluorohexyl bromide, a perfluorooctane, a perfluoropentane, a perfluorohexane, a perfluorodecalin, a perfluorotributylamine, a salt of perfluorotributylamine, a perfluorotriisopropylamine, a salt of perfluorotriisopropylamine, a perfluoro-crown ether containing 12 crown ethers, a perfluoro-crown ether containing 15 crown ethers, and a perfluoro-crown ether containing 18 crown ethers.

12. The implant of claim 1, wherein the oxygen scavenger induces transient hypoxia in the tissue surrounding the implant upon implantation.

13. The implant of claim 12, wherein the transient hypoxia in the tissue surrounding the implant induces expression of pro-angiogenic growth factors in the tissue surrounding the implant.

14. A method of preparing a composition for repairing tissue injury with enhanced regenerative capacity comprising combining a tissue graft with a biocompatible oxygen scavenger.

15. The method of claim 14, wherein the tissue graft is of a biological origin.

16. The method of claim 15, wherein the tissue graft of the biological origin is selected from the group consisting of a cortical bone, a cancellous bone, a demineralized bone, a partially demineralized bone, a connective tissue, a tendon, a pericardium, dermis, a cornea, a dura matter, fascia, a heart valve, a ligament, a capsular graft, a cartilage, collagen, a nerve, a placental tissue, and combinations thereof.

17. The method of claim 14, wherein the tissue graft is of a synthetic origin.

18. The method of claim 17, wherein the tissue graft of the synthetic origin is selected from the group consisting of a metal, a thermoplastic, an elastomer, a polymer, a mineral, an organic mineral, and combinations thereof

19. The method of claim 14, wherein the biocompatible oxygen scavenger is of a biological origin.

20. The method of claim 19, wherein the biocompatible oxygen scavenger of the biological origin is selected from the group consisting of a heme-based formulation, a hemoglobin-based formulation, and a myoglobin-based formulation.

21. The method of claim 14, wherein the biocompatible oxygen scavenger is of a synthetic origin.

22. The method of claim 21, wherein the biocompatible oxygen scavenger of the synthetic origin is a perfluorocarbon.

23. The method of claim 22, wherein the perfluorocarbon is at least one of perfluorooctyl bromide, perfluorohexyl bromide, perfluorooctane, perfluoropentane, perfluorohexane, perfluorodecalin, perfluorotributylamine, a salt of perfluorotributylamine, a perfluorotriisopropylamine, a salt of perfluorotriisopropylamine, a perfluoro-crown ether containing 12 crown ethers, a perfluoro-crown ether containing 15 crown ethers, and a perfluoro-crown ether containing 18 crown ethers.

24. The method of claim 14, wherein the biocompatible oxygen scavenger of the composition is deoxygenated.
